(19) United States
(12) Patent Application Publication
(10) Pub. No.: US 2004/0127855 A1
(43) Pub. Date: Jul. 1, 2004

(54) Hemostasis valve

(75) Inventor: Lee A. Core, Cambridge, MA (US)

Correspondence Address: HALE AND DORR, LLP, 60 STATE STREET, BOSTON, MA, 02109

(73) Assignee: NMT Medical, Inc., Boston, MA

(21) Appl. No.: 10/682,184

(22) Filed: Oct. 9, 2003

Related U.S. Application Data
(60) Provisional application No. 60/417,705, filed on Oct. 10, 2002.

(51) Int. Cl. A61M 5/178; A61M 5/00; A61M 5/14; B29C 33/40

(52) U.S. Cl. 604/167.04; 604/167.01; 604/256; 264/219

(57) ABSTRACT

A hemostasis valve includes a single gland with multiple offset longitudinal slits that do not extend through the gland completely. The multiple slits form a complex pathway for the guide dilator/catheter and thus a better seal. The offset slits are joined by a lateral cut in the gland. When a guide wire or dilator/catheter is introduced into the introducer, the gland deforms sufficiently to allow the guide wire or other device to move through the first slit, the lateral cut and the second slit. This complex pathway or slit provides a larger sealing surface thereby creating a better seal. As a result, effective seals may be formed around relatively large, as compared to the inner diameter of an introducer sheath, catheters.

### CROSS REFERENCE TO RELATED APPLICATION

This application claims the benefit of U.S. Provisional Application 60/417,705, filed on Oct. 10, 2002, which is incorporated herein by reference.

### BACKGROUND OF THE INVENTION

1. Field of the Invention

The present invention relates generally to introducer sheaths for use in procedures requiring vascular access. More specifically, the present invention relates to hemostasis valves that may be used in such an introducer.

2. Description of Related Art

Vascular introducer sheaths are used in a wide variety of diagnostic and therapeutic vascular procedures, such as angiography, angioplasty, embolization and any other procedure requiring vascular access. Vascular access systems typically include an introducer sheath which is used with a guide wire and a dilator. The sheath usually includes some kind of hemostatic valve to inhibit blood loss as various diagnostic and therapeutic catheters are introduced into the vasculature and manipulated during the clinical procedure. Often, a guide wire is introduced early in the clinical procedure and a series of catheters are inserted into the vasculature system over the guide wire. Accordingly, the valve must form an adequate seal when a guidewire is present in the introducer as well as when catheters are advanced over the guide wire.

Since a hemostatic valve is intended to minimize blood loss during a catheterization procedure, the valve, or gland, must form an adequate seal around the guide wire, dilator or catheter, which is introduced into the vasculature. However, the valve cannot form such a tight seal that the gland significantly restricts the movement of the guidewire or catheter during the procedure. There have been many attempts to balance these competing design goals. Some designs use multiple glands to form the seal. However, the use of multiple glands to form the seal can increase the cost of the introducer sheath because of the increase in the number of parts and also the increased number of manufacturing steps required to construct the device.

Another alternative has been to use a single gland with a single slit through the gland. To form an effective seal the gasket has to be of a sufficient thickness so that the valve will inhibit the flow of blood and maintain its integrity during use. If a single slit is used, the seal around the guide wire may not be complete and blood can leak out at the edges of the slit.

### BRIEF SUMMARY OF THE INVENTION

The present invention overcomes the disadvantages of the prior art by providing, in an exemplary embodiment, a single gland with multiple offset longitudinal slits that do not extend through the gland completely. The multiple slits form a complex pathway for the guide wire and thus a better seal. The offset slits are joined by a lateral cut in the gland. When a guide wire or dilator/catheter is introduced into the introducer, the gland deforms sufficiently to allow the guidewire to move through the first slit, the lateral cut and the second slit. This results in a greater sealing surface and therefore a better seal. The invention allows sealing around relatively large diameter catheters as compared to the sheath's inner diameter.

In one embodiment the gland is constructed using a single piece of elastomeric material with the lateral slice formed by cutting through a side or circumferential edge of the material approximately halfway between the top and bottom surfaces. Offset slits are then made on the top face and the bottom face a sufficient distance apart to provide a suitable cut through the material. In another embodiment, the gland is molded as a single piece with offset slits molded into the gland. In this embodiment, the gland may be molded with an insert that forms the complex path. After forming the gland, the insert may be removed with the complex pathway formed in the gland.

### BRIEF DESCRIPTION OF THE DRAWINGS

FIG. 1 is a plan view of a vascular access system of the present invention including an introducer sheath and a dilator;

FIG. 2 is a cross section of the hemostasis valve assembly of the present invention;

FIG. 2a is an alternative embodiment of the hemostasis valve of the present invention with a lateral slice extending from a circumferential edge of the valve to a longitudinal slit;

FIG. 3 is an alternative embodiment of the hemostasis valve of the present invention;

FIG. 4 is an isometric perspective view of the hemostatic valve of the present invention;

FIG. 5 is an illustration of the hemostatic valve as a guide wire is inserted through the valve.

### DETAILED DESCRIPTION

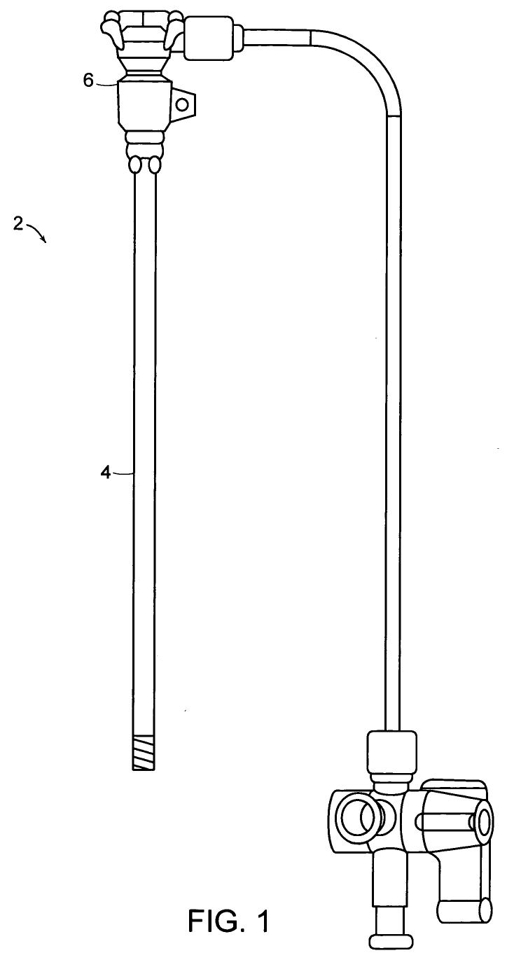

The present invention provides a hemostatic introducer sheath with an improved valve structure. As illustrated in FIG. 1, an introducer sheath 2 has an elongated shaft 4 and a hemostatic valve assembly 6. A dialator, typically used with the introducer sheath, is not illustrated. The hemostatic valve is connected to the proximal end of the shaft using conventional techniques.

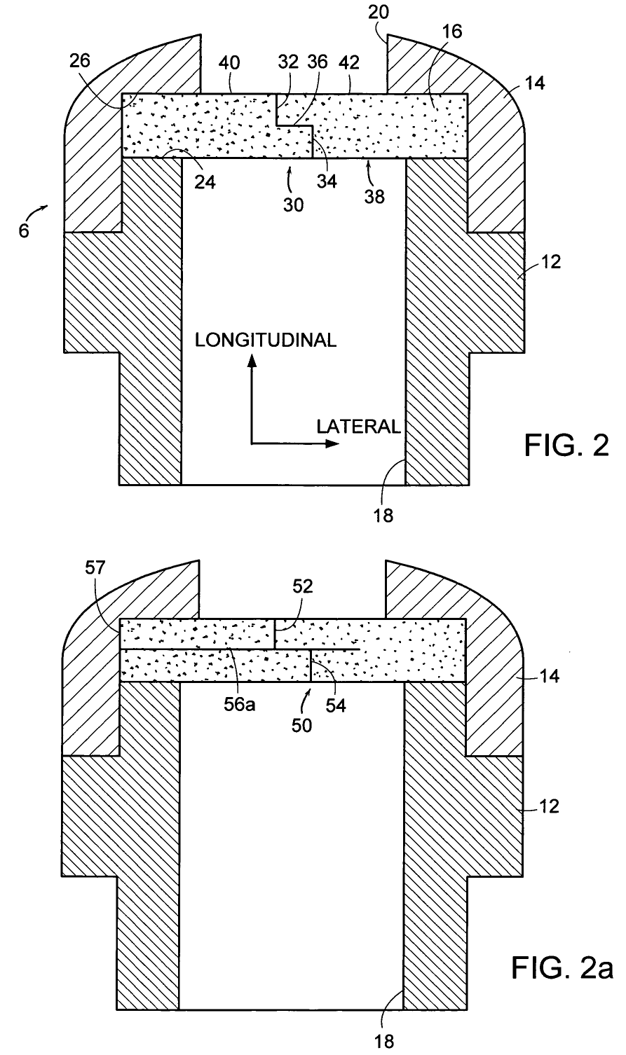

The hemostatic valve assembly includes a hub, a cap and a gland disposed between the hub and the cap. The gland is described in detail in FIGS. 2-4. The hub of the hemostasis assembly may include a side port for connection to a subassembly for flushing or injecting fluid. The size of the shaft 4 may have an outside diameter ranging from 3 Fr. to 16. Fr. and a length ranging from 10-75 cm. The distal tip of the sheath may have a conventional taper to reduce trauma to tissue as the sheath is introduced into a blood vessel.

Referring now to FIG. 2, which illustrates a cross sectional view of an exemplary embodiment of the hemostatic valve assembly of the present invention, a hemostasis valve assembly 6 includes a hub 12, a cap 14 and a gland 16 disposed between the hub and the cap. For the purposes of simplicity, the side port is not illustrated in FIG. 2. The cap 14 is disposed on the hub 12 in a manner well known in the art, such as with adhesive or a snap fit.

The hub includes an inner lumen 18 and the end cap includes an aperture 20. When the cap is disposed on the hub, the inner lumen and the aperture provide a centrally located lumen through the valve assembly. The hub and cap may have conventional dimensions and may be formed of any material using any known manufacturing techniques.

The hub 12 includes an end face surface 24 which faces gland 16. Similarly, the cap 14 has an inwardly facing surface 26 which faces the gland 16. The gland is disposed between the two surfaces 24 and 26. In some embodiments, the annular edges of the gland may be sealed to the hub and/or the end cap in a manner known to prevent leaks.

The gland may be a flat disc shape as disposed in the hub. The gland may be formed of a variety of elastomeric materials such as silicone, latex or other suitable material. Preferably the gland material has a durometer in the range of 30-60A. The thickness of the gland may range from 0.040-0.130 inches and may have an outside diameter ranging from 0.125 to 0.625 inches to fit securely between the cap and the hub. The gasket may be punched out of a single sheet of material or may be molded using conventional techniques.

The configuration of slits forms one aspect of the present invention. That is, the slit that is formed in the gland is not a simple slit through the gland. Rather, a plurality of slits are used that are offset from one another and connected by a laterally oriented slit. As illustrated in FIG. 2, a complex slit, indicated generally by 30, is formed by two longitudinal slits 32, 34 which do not extend entirely through the gland. The longitudinal slit 32 extends from an outside or outwardly facing (i.e., proximal) surface 40 of the gland to a location approximately midway within the gland. The longitudinal slit 34 extends from an inside or inwardly facing (i.e., distal) surface 38 of the gland to a location approximately midway within the gland. A lateral slit 36 joins the inside edges of the longitudinal slits 32 and 34. Thus, a complex slit is formed in the gland which allows a catheter and/or dilator guide wire to extend through the gland via slits 34, 36, and 32 (from inside toward outside).

In a particular embodiment, a gland can be 0.070 inches thick and have a diameter of 9/16^{th} inches. The longitudinal slits are parallel and extend a distance of 0.110 inches and are 0.050 inches apart. The lateral slit may have a 0.225-inch width and may cut through the middle (approximately) of the gland. In a particular manufacturing method, a razor blade or other suitable cutting instrument may be used to create the slits. When creating the lateral cut, an operator may secure the gland and slice the gland along the edge toward the center until the longitudinal slits are joined. For example, referring to FIG. 2a (where like elements are referenced using like numerals), a lateral slice or cut 56a is made extending from a side or circumferential edge 57 of the gland to a position that connects longitudinal slits 52 and 54. Slice 56a may extend to the slit farthest from the side (or beyond) so long as slits 52 and 54 are connected. In other embodiments, lateral slice 56a may be made before cutting slits 52 and 54. In these embodiments, the razor or other device used to cut slice 56a may serve as a backstop for slits 52 and 54.

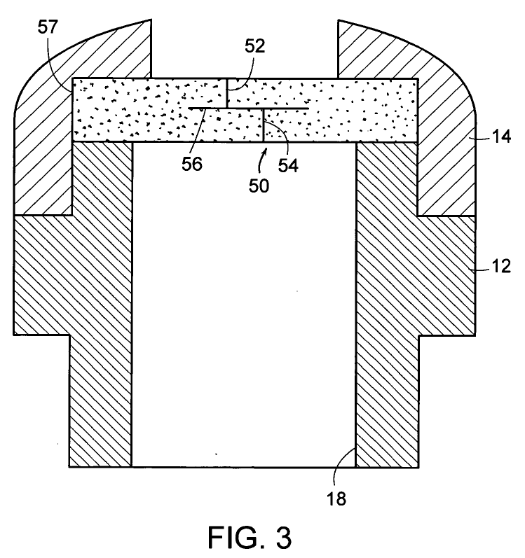

In another embodiment of the invention, in which like elements are referenced using like numerals, the gland 50 may have a lateral slit that extends beyond the offset longitudinal slits. With specific reference to FIG. 3, the longitudinal slits 52 and 54 are formed in an offset manner consistent with the first embodiment. In this embodiment, a lateral slit 56 extends beyond the longitudinal slits. The length that the lateral slit extends beyond the slits can be as little as 0.005 inches or as much as half the diameter of the gland. The dimension chosen for the longitudinal slit may be selected to enhance the seal or to facilitate the exchange of catheters on a guide wire. Of course, the distance between the longitudinal slits can also be varied to adjust the amount of seal and to facilitate the exchange of dilators/catheters on the guide wire.

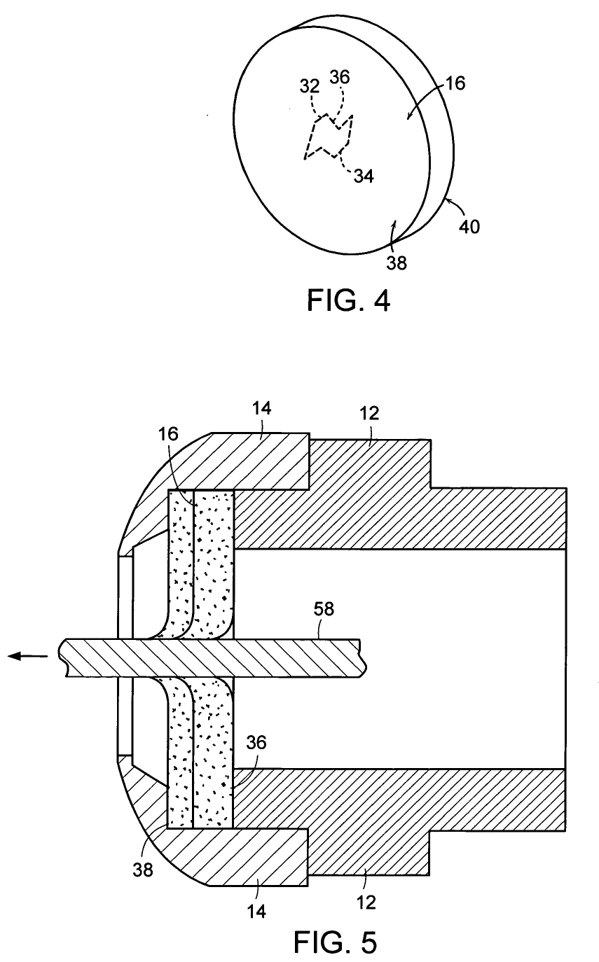

FIG. 4 illustrates an isometric view of the gland according to another embodiment of the invention. To facilitate an understanding of the invention, FIG. 4 has been labeled consistently with the numerals used in FIG. 2. Of course, the orientation of the slits may be modified so that they are not parallel without departing from the scope of the invention. In addition, the intersections created between the slits are not limited to being right angles. For instance, any suitable angle (e.g., 100 degrees) may be utilized.

FIG. 5 illustrates the gland of the present invention with a guide wire 58 inserted through the complex slit. As illustrated the gland deforms to allow the guide wire through the complex slit. Since the gland is formed from an elastomeric material, it will deform sufficiently to allow the guide wire and catheter through the complex slit while maintaining an adequate seal.

Having described preferred embodiments of the invention, it should be apparent that various modifications may be made without departing from the spirit and scope of the invention.

What is claimed is:

1. A gland for use in a hemostasis valve assembly comprising;
a self-sealing, one piece gland having an inwardly facing surface, an outwardly facing surface located proximally from said inwardly facing surface, a first slit formed in said inwardly facing surface extending in a longitudinal direction toward but terminating before said outwardly facing surface, a second slit formed in said outwardly facing surface extending in said longitudinal direction toward but terminating before said inwardly facing surface in a plane offset from said first slit, and a third slit formed in a plane substantially parallel to at least one of said inwardly facing and outwardly facing surfaces extending laterally to connect said first slit with said second slit.

2. The gland of claim 1, wherein said third slit extends from a circumferential edge of said gland to a position beyond both of said first and said second slits.

3. The gland of claim 1, wherein said first slit is substantially parallel to said second slit.

4. The gland of claim 1, wherein said third slit is substantially perpendicular to at least one of said first and said second slits.

5. The gland of claim 1, wherein said third slit is substantially perpendicular to both of said first and said second slits.

6. The gland of claim 1, wherein said third slit extends from said first slit to said second slit without extending to a circumferential edge of said gland.

7. The gland of claim 1, wherein said third slit extends from a position between a circumferential edge of said gland and said first slit to a position between an opposite circumferential edge of said gland and said second slit without extending to either of said circumferential edge and said opposite circumferential edge.

8. The glad of claim 1, wherein each of said first, second and third slits is defined by a first surface of material in abutting contact with a second surface of material.

9. A method of manufacturing a hemostasis valve gland, said method comprising the steps of:
cutting a lateral slit into an elastomeric material extending from a circumferential edge into said material;
cutting a first longitudinal slit into said material extending from a top surface of said material to said lateral slit;
cutting a second longitudinal slit into said material extending from a bottom surface of said material to said lateral slit thereby forming a complex slit through which a device may be advanced.

10. A method of manufacturing a hemostasis valve gland, said method comprising the steps of:
cutting, using a first cutting device, a lateral slit into an elastomeric material extending from a circumferential edge into said material;
cutting, using a second cutting device, a first longitudinal slit into said material extending from a top surface of said material toward said first cutting device;
terminating said cutting of said first longitudinal slit when said second cutting device contacts said first cutting device;
removing said second cutting device from said material; and
removing said first cutting device from said material.

11. The method of claim 10, further comprising:
subsequent to said cutting of said first longitudinal slit and prior to said removing of said first cutting device, cutting a second longitudinal slit into said material extending from a bottom surface of said material; and
terminating said cutting of said second longitudinal slit when said first cutting device is contacted.

12. A method of manufacturing a hemostasis valve gland, said method comprising the steps of:
positioning an insert into a mold, wherein said insert comprises a first planar member extending in a longitudinal direction, a second planar member offset from said first planar member and extending in a direction substantially parallel to said longitudinal direction, and a third planar member extending in a direction substantially perpendicular to said longitudinal direction connecting said first and said second planar members;
pouring an elastomeric material into said mold; and
removing said insert from said elastomeric material.
